(19) United States
(12) Patent Application Publication
(10) Pub. No.: US 2023/0277390 A1
(43) Pub. Date: Sep. 7, 2023

(54) Intimate Pad

(71) Applicants: Stefania Alejandra Rocha, South Jordan, UT (US); Colby Tyler Robertson, South Jordan, UT (US)

(72) Inventors: Stefania Alejandra Rocha, South Jordan, UT (US); Colby Tyler Robertson, South Jordan, UT (US)

(21) Appl. No.: 17/684,276

(22) Filed: Mar. 1, 2022

(51) Int. Cl. A61F 13/15

(52) U.S. Cl. CPC A61F 13/15203; A61F 2013/15056; A61F 2013/15243; A61F 2013/51033

(57) ABSTRACT

An intimate pad for cathing fluids during sexual encounters. The intimate pad comprises one or more superabsorbent polymer layers and a non-porous material layer fixed together in a rectangular or square shape. Further comprising an optional flash-dry or equivalent technology that may be inserted between a superabsorbent polymer layer and a non-porous layer for extra liquid absorption. Further comprising adhesive placed on the bottom layer capable of temporarily sticking to the desired surface for more effective liquid absorption. Further comprising printed graphics that would be imprinted on one or multiple layers. Further comprising scented particles incorporated into one of the layers as an aphrodisiac. Further comprising a corner of the pad shall be folded in a way for easy open and placement of the pad.

### FIELD OF THE INVENTION

Embodiments of the present disclosure generally relate to absorbent pads used for other activities such as a dog training pad, incontinence pad, or bedwetting pad. The named pads include multiple layers of surface protection and enhanced absorption of fluids.

### BACKGROUND

Disposable absorbent pads have been in use for many years over a variety of use cases. Most situations involving human fluids have already been covered such as urine pads, incontinence pads, or period pads. However, these use cases do not cover the absorption of intimate fluids during sexual encounters. Fluids not limited to but such as semen, cum, menstrual blood, lubricant, or drool that may fall onto surfaces that need to be cleaned immediately to prevent material or surface damage.

Accordingly, there exists a need to provide a disposable absorbent pad that catches body fluids during sexual encounters. Ideally, this pad can be used in several locations including but not limited to on a bed, on a couch, or on the floor. This pad should be easy to access, lightweight and unnoticeable during a sexual encounter. Lastly, the pad should catch and absorb these fluids for easy, quick clean-up and disposal.

### SUMMARY

One objective of the present disclosure is to provide an intimate pad with liquid absorption for sexual encounters.

Another objective of the present disclosure is to provide an intimate pad with liquid absorption that is portable, can be used on multiple surfaces, and easy disposal.

In accordance with one aspect of at least one embodiment of the present disclosure, the intimate pads comprise 1) a top layer made from super absorbent polymer; 2) a bottom layer made from a non-porous material.

The foregoing and other objects, features, and advantages of the present invention are more readily apparent from the detailed description of the preferred embodiments set forth below, taken in conjunction with the accompanying drawings.

### DESCRIPTION

The following description is made for the purpose of illustrating the overall principles of the invention, yet should not be taken in a limiting sense. The following inventive features can be used independently from each other or as a combination. The scope of the invention is best defined by the following claims.

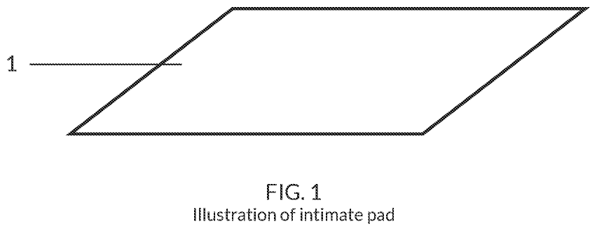

Broadly, embodiments of the present disclosure generally provide intimate pads with liquid absorption for sexual encounters. In accordance with one embodiment of the present disclosure as shown by FIG. 1, an intimate pad 1 with liquid absorption for sexual encounters.

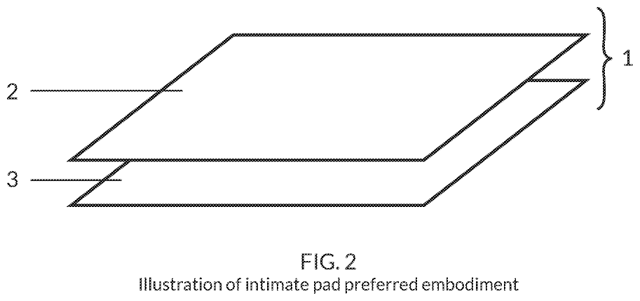

Another embodiment of the present disclosure, shown in FIG. 2, comprises a top layer 2 made from superabsorbent polymer (SAP) and a bottom layer 3 made from a non-porous material.

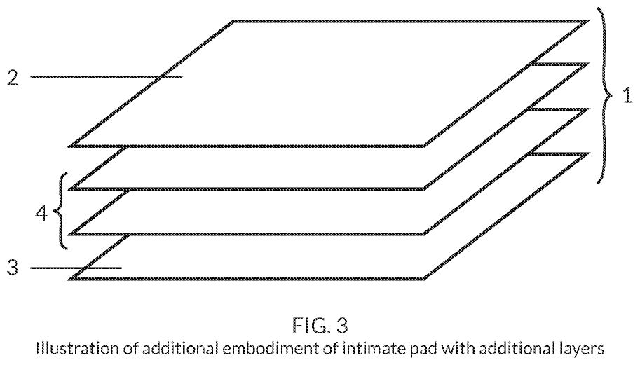

In yet another embodiment of the present disclosure, shown in FIG. 3 comprises a top layer 2 made from superabsorbent polymer (SAP), inner layer(s) 4 additional super absorption polymer (SAP), and bottom layer 3 made from a non-porous material.

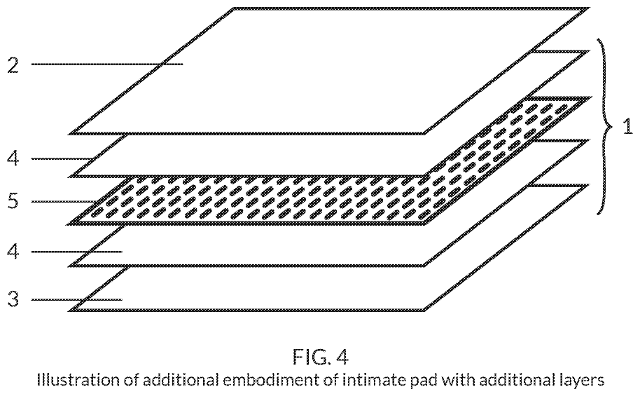

In yet another embodiment of the present disclosure, shown in FIG. 4 comprises a top layer 2 made from superabsorbent polymer (SAP), inner layer(s) 4 additional super absorption polymer (SAP), a middle layer 5 flash-dry or equivalent technology, and bottom layer 3 made from a non-porous material.

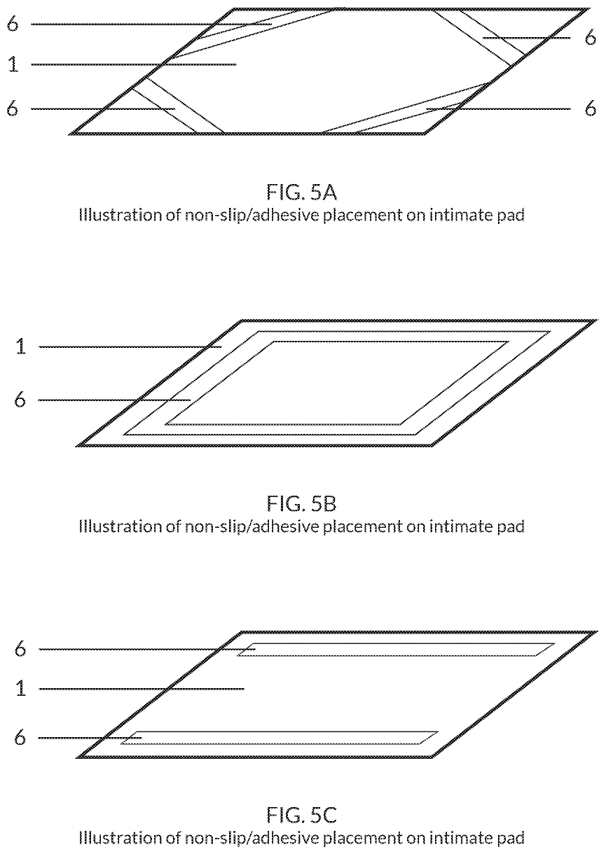

FIGS. 5A, 5B, and 5C introduce another embodiment of the present disclosure, with optional adhesive or non-slip back 6 such as but not limited to non-slip plastic or rubber or adhesive tape attached to the bottom of bottom layer 3. The adhesive or non-slip back may be placed in several different patterns included in but not limited to FIGS. 5A, 5B, and 5C for the intimate pad 1 to provide temporary adhesion to surfaces.

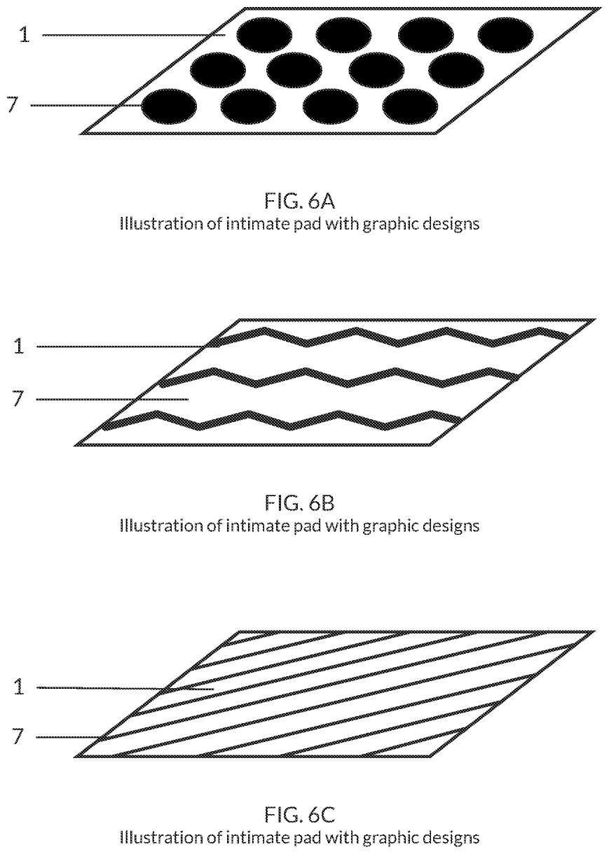

FIGS. 6A, 6B, and 6C show another embodiment of the present disclosure, which includes graphic designs 7 placed on top layer 2 but not limited to visuals seen on FIG. 6A, 6B, or 6C.

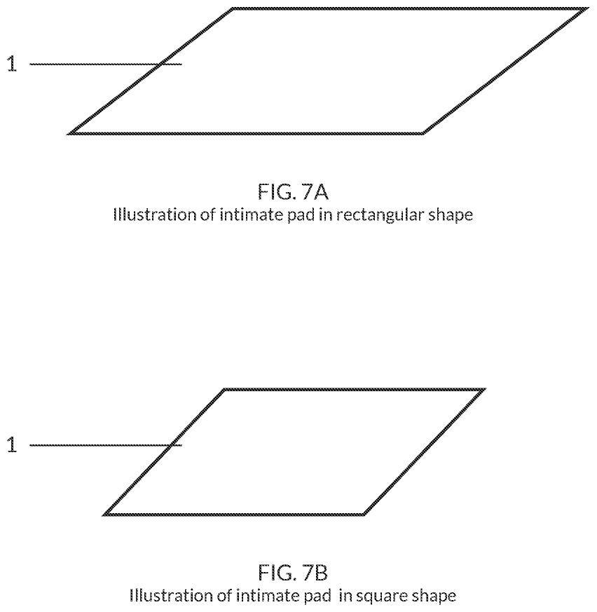

In yet another embodiment of the present disclosure, the intimate pad 1 is shown with a rectangular as seen on FIG. 7A or a square shape as seen on FIG. 7B.

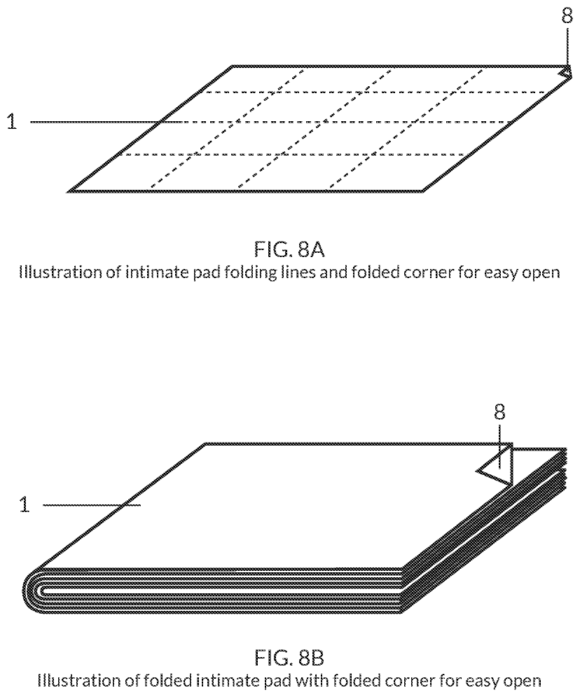

In yet another embodiment of the present disclosure, FIGS. 8A and 8B embody the intimate pad 1 with a folded corner for easy open use when intimate pad 1 is folded. Folding patterns like but not limited as seen on FIG. 8A.

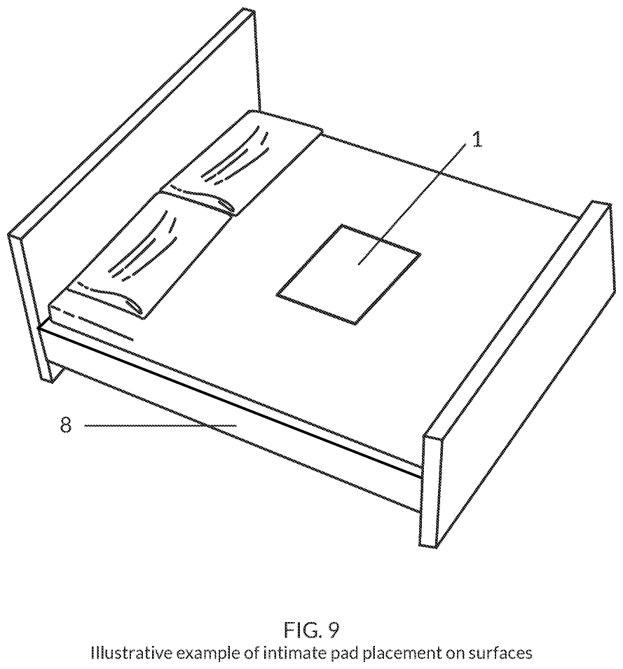

FIG. 9 embodies how intimate pad 1 with liquid absorption for sexual encounters will be placed on one of several surfaces such as but not limited to beds, couches, and floors.

What is being claimed is:

1. An intimate pad comprising:
a. a top layer made of but not limited to a superabsorbent polymer; and
b. a bottom layer made of but not limited to a non-porous material.

2. An intimate pad as in claim 1, wherein said bottom layer further comprises of but not limited to an adhesive that is capable of temporality sticking one side of the bottom layer to a surface.

3. An intimate pad as in claim 1, wherein said top layer comprises of but not limited to printed graphics.

4. An intimate pad as in claim 1, wherein scented particles are incorporated into the said layers.

5. An intimate pad as in claim 1, wherein a corner may be folded for easy open to use.

6. An intimate pad comprising:
a. a top layer made of but not limited to a superabsorbent polymer;
b. an additional layer of absorbent made from at least one of but not limited to a superabsorbent polymer and flash-dry or equivalent technology is placed between the top layer and the bottom layer; and
c. a bottom layer made of but not limited to a non-porous material.

7. An intimate pad as in claim 6, wherein said bottom layer further comprises of but not limited to an adhesive that is capable of temporality sticking one side of the bottom layer to a surface.

8. An intimate pad as in claim 6, wherein said top layer comprises of but not limited to printed graphics.

9. An intimate pad as in claim 6, wherein scented particles are incorporated into the said layers.

10. An intimate pad as in claim 6, wherein a corner may be folded for easy open to use.
